SEQUENCE LISTING (12 sequences)

SEQ ID NO: 1 - DNA, 2913 bp, Artificial Sequence
  PSA promoter and enhancer region
agcttggggctggggagcctcccccaggagccctataaaaccttcattccccagaactccgcccctgccctgctggcacccagaggctgaccaaggccctctccatgctgctggaggctgtacaaccccctcccacacccagagctgtggaaggggagggagagctagtacttgctgttctgcaattactagatcaccctggatgcaccaggccctgtggctcatggagacttcatctaggggacaaaggcagaggagacatgcccaggatgaaacagaaacagggggtgggtacgatccccgattcttcatacaaagcctcacgtgcctagatcctttgcactccaagacccagtgtgccctaagacaccagcactcaggagattgtgagactccctgatccctgcaccgctctgagaccagaaactagaacttttattcctcatgctcctgaaatagatgtcttggcatttagtacattcttttccttgcactcccaacccagaatccagctccacagatacattgctactgtcatcataaaaagatctggtggagataaggctgatgctggcacagtgcctcgtatctgggagactcacttgggagagcaatagacttgggaaaccataagttcaggtccctgtgccctccacccagaagcccttgagatttcatggatggtgacatatggccattctctagcacttttgagtcctggatccctatgattccaggctccctgtctgacactaggcctcagcctggcaccatgcaggccactcgcaccagaggaggatgggcagctctacggttggcaggtggctggagaggcacgctgcagggatagtcacagcaacatgacgtcatggtgctggccacaccctcagagtgtgatgctggatgatgagtggatgacacccaaggcctctggggcatctttcatgctcagattgtgcttgcccaggggaccttcatggagctagaagggctggtgataccctcaagtggagtaaggattaggtggcaagatggaagaatgggcaaccctcgatcctcagactcttgaggaatcaagagatcccattactatcaccccaaaccctggacctaatggttccagcctctgccttcaggggccaaagagccttaagccacaaatatacccagaactctacccctcaggattccagcaccttcttcctgaggatatgagattcttaggccattcccacatcagtacctcgggagctgggaccttaccagtctcctccctcattgacctaagagttcggaactgacactttccctcccccagtaccttcacatccagcctcttcctcctttgaaattcaagagggtggaccgactcctcactcaaacccagaagttctgatccccagccatgccccttcgggatcctgagcgctgccttattctgggtttggcagtggagtgctgccagacacagtcgatcgggacctagaaccttggttaggcataaagaagcaggatgtgatagaagaagtatttaatggtggaacgttgagactgtcctgcagacaagggtggaaggctctggctgaacagcgttgggaggcaattctccatggttctgtcacgtatctgtgtgtcttctgagcaaagacagcaacacctttttttctggattgttgtttcaaggatgdttgtaaagcaggcatccttgcaagatgatatctctctcagatccaggcttgcttactgtcctagataataaagataatgtctcttacaacagatttgtttactgtcaaggacaatcaatacaatatgttcctccagagtaggtctgttttcaatccaagatcatgaagataatatcttcatcagagacaaaggctgagcaggtttgcaagttgtcccagtataagattgaggattcctaatctcaggtttctcaccagtggcacaaaccccgtgtgcacagcatccacctagactgctctggtcaccctacaagatttggggggggcaaggtgtactaatgtgaacatgaacctcatgctgtctgctaagctgtgagcagtaaaagcctttgcctctgactcaggagtctcatggactctgccaacattcacaaaactctggaaagttagcttatttgtttgcaagtcagtaaaatgtcagccccttcagagtaactgacaaacaggtgggcactgagactgcactggccagctgggaatagagataggaggggacccagctggatgcagtgggcagtgggggtcatagagtcaagagtgtacagaatacaatggggtcctagtatcatggtggaggtcagaaagagccctaaaagagagggtcaaggtaggaggttagtgaaggtccacctccaccctctccaggacagggacatcaggccacaattaatttctctgcagttggtgagtggtcatggtctctggagtccccagcatccagagtgtccctggtctagtggtcccccctttctgagccacagccactttctccatcaaatgaggccagtaatacccatcccatagtgatgctgtgaggatgagatgagcatctgtaagtgctgaagataatccctgacacatcccaagcattcagcagtgcaagcatacacttacacggcactccccagagccaggcatgtgctggtgcctcatacacgtgaccacatttgatcgtcacaatgaccctgtgagggagactgtgcaacagaggactgaccttgctcaaagacctcaggcgtttcccctcagagcctgagaggtcatctctttttttttttttttttcctttctttctttttcttttccatttctttttctttgcaagaggtcatctctaatgctttggaatatcctgccagattagagtccctttgttcacctgaag

SEQ ID NO: 2 - DNA, 96 bp, Artificial Sequence
  siRNA coding sequence
gttttatagggctcctgggggaggctccccagccccaagctgaagcagcacgacttcttcattttgctgaagaagtcgtgctgcttcaaataaagc

SEQ ID NO: 3 - DNA, 91 bp, Artificial Sequence
  siRNA sequence targeting GFP
atgaattcaaaaaagctttatttgaagcagcacgacttcttcagcaaaatgaagaagtcgtgctgcttcagcttggggctggggagcctcc

SEQ ID NO: 4 - DNA, 47 bp, Artificial Sequence
  JNK target sequence
gatcagtggaataaagttatttttgcaataactttattccactgatc

SEQ ID NO: 5 - DNA, 47 bp, Artificial Sequence
  PI3K target sequence
aagcaagttcacaattacccatttgctgggtaattgtgaacttgctt

SEQ ID NO: 6 - DNA, 28 bp, Artificial Sequence
  primer
atctcgagccgagaaattaattgtggcg

SEQ ID NO: 7 - DNA, 88 bp, Artificial Sequence
  primer
atgaattctttattaagcttgaagcagcacgacttcttcagcaaaatgaagaagtcgtgctgcttcagcttggggctggggagcctcc

SEQ ID NO: 8 - DNA, 86 bp, Artificial Sequence
  primer
atgaattctttattgatcagtggaataaagttattcgaaaataactttattttattccactgatcgcttggggctggggagcctcc

SEQ ID NO: 9 - DNA, 81 bp, Artificial Sequence
  primer
atgaattctttattaagcaagttcacaattacccacgaatgggtaattgtgaacttgcttgcttggggctggggagcctcc

SEQ ID NO: 10 - DNA, 21 bp, Artificial Sequence
  JNK target sequence
gatcagtggaataaagttatt

SEQ ID NO: 11 - DNA, 21 bp, Artificial Sequence
  PI3K target sequence
aagcaagttcacaattaccca

SEQ ID NO: 12 - DNA, 21 bp, Artificial Sequence
  GFP  target sequence
tgaagcagcacgacttcttca

(19) United States
(12) Patent Application Publication
(10) Pub. No.: US 2008/0131940 A1
(43) Pub. Date: Jun. 5, 2008

(54) Target Cell-Specific Short Interfering Rna and Methods of Use Thereof

(76) Inventors: Robert Chiu, Tustin, CA (US); Jun Song, Los Angeles, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/570,423

(22) PCT Filed: Jun. 22, 2005
(86) PCT No.: PCT/US05/22290

§ 371 Date: Nov. 16, 2007

Related U.S. Application Data
(60) Provisional application No. 60/583,176, filed on Jun. 25, 2004.

(51) Int. Cl. C12P 19/34; C07H 21/02; C12N 15/63; C12N 5/00; C12N 15/87

(52) U.S. Cl. 435/91.3; 536/23.1; 435/320.1; 435/325; 435/463

(57) ABSTRACT

The present invention provides nucleic acids that include a nucleotide sequence that encodes an siRNA, which nucleotide sequence is operably linked to a target cell-specific promoter RNA polymerase II promoter. The present invention further provides vectors, including expression vectors, which include a subject nucleic acid; and host cells that harbor a subject nucleic acid or a subject expression vector. The present invention further provides methods of modulating (e.g., reducing) expression of a gene in a target cell-specific manner, the methods generally involving introducing into a cell a subject expression vector.

### CROSS-REFERENCE

This application claims the benefit of U.S. Provisional Patent Application No. 60/583,176, filed Jun. 25, 2004, which application is incorporated herein by reference in its entirety.

### FIELD OF THE INVENTION

The present invention is in the field of short interfering RNA (siRNA), and in particular the use of siRNA to control gene expression in a target cell-specific manner.

### BACKGROUND OF THE INVENTION

Small interfering RNAs (also known as “short interfering RNAs” or “siRNA”) are short double-stranded RNA (dsRNA) fragments that elicit a process known as RNA interference (RNAi), a form of sequence-specific gene silencing. Zamore, Phillip et al., Cell, 101:25-33 (2000); Elbashir, Sayda M., et al., Nature 411:494-497 (2001). siRNAs are assembled into a multicomponent complex known as the RNA-induced silencing complex (RISC). The siRNAs guide RISC to homologous mRNAs, targeting them for destruction. Hammond et al., Nature Genetics Reviews 2:110-119 (2000).

### Literature

Shinagawa and Ishii (2003) Genes Dev. 17:1340-1345; U.S. Patent Publication No. 20040115815; Xia et al. (2002) Nat. Biotechnol., 20: 1006-1010; Hara et al. (1989) J. Lab. Clin. Med., 113: 541-548; Wang et al. (1979) Invest. Urol., 17: 159-163; Chan et al. (1987) Clin. Chem. 33:1916-1920; Martin et al. (2004) Cancer Res. 64:347-355; Riegman et al. (1991) Mol. Endocrinol., 5: 1921-1930; Pang et al. (1995) Hum. Gene Ther., 6: 1417-1426; Louie et al. (2003) Proc. Natl. Acad. Sci. USA., 100: 2226-2230; Pang et al. (1997) Cancer Res., 57: 495-499; Yu et al. (2001) Cancer Gene Ther. 8: 628-635; Rubinson et al. (2003) Nat. Genet. 33: 401-406; Lois et al. (2002) Science, 295: 868-872; Tiscornia et al. (2003) Proc. Natl. Acad. Sci. USA, 100: 1844-1848; Kuan et al. (1999) Neuron, 22: 667-676; Beresford et al. (2001) Interferon Cytokine Res., 21: 313-322; Engedal et al. (2002) Oncogene, 21: 1017-1027.

### SUMMARY OF THE INVENTION

The present invention provides nucleic acids that include a nucleotide sequence that encodes an siRNA, which nucleotide sequence is operably linked to a target cell-specific, RNA polymerase II promoter. The present invention further provides vectors, including expression vectors, which include a subject nucleic acid; and host cells that harbor a subject nucleic acid or a subject expression vector. The present invention further provides methods of modulating (e.g., reducing) expression of a gene in a target cell-specific manner, the methods generally involving introducing into a cell a subject expression vector.

### Features of the Invention

The present invention features an isolated nucleic acid comprising, in order from 5′ to 3′ and in operable linkage, a target cell-specific RNA polymerase II promoter, and a nucleotide sequence encoding a short interfering RNA. In some embodiments, the nucleic acid further comprises an inducible promoter 5′ of the target cell-specific RNA polymerase II promoter. In some embodiments, the target cell-specific promoter directs transcription in cancer cells. In some embodiments, the cancer cells are prostate cancer cells. In some embodiments, the cancer cells are breast cancer cells. In some embodiments, the siRNA reduces expression of a gene involved in cell proliferation. In some embodiments, the target cell-specific promoter directs transcription in CD4⁺ T cells. In some embodiments, the target cell-specific promoter directs transcription in human immunodeficiency virus-1 (HIV-1)-infected cells. In some embodiments, the siRNA reduces expression of HIV-1.

The present invention further features a recombinant expression vector comprising a subject nucleic acid, where the nucleic acid comprises, in order from 5′ to 3′ and in operable linkage, a target cell-specific RNA polymerase II promoter, and a nucleotide sequence encoding a short interfering RNA. The present invention features a composition comprising a subject recombinant expression vector.

The present invention further features a genetically modified host cell comprising a subject recombinant expression vector. In many embodiments, the genetically modified host cell is a eulcaryotic cell. In some embodiments, the genetically modified host cell is an in vitro cell. In some embodiments, the genetically modified host cell is a cell of a transgenic non-human animal that comprises as a transgene the subject nucleic acid.

The present invention further features methods of reducing expression of a target gene in a target cell. The methods generally involve introducing a subject recombinant expression vector into the target cell, where the encoded siRNA is specific for the target gene, and reduces expression of the target gene. In some embodiments, the target gene is an endogenous gene. In some embodiments, the target gene is an exogenous gene. In some embodiments, the target gene encodes a product that controls cell proliferation. In some embodiments, the target gene is a gene of an intracellular pathogen. In some embodiments, the target gene is a viral gene. In some embodiments, the target cell is a eukaryotic cell. In some embodiments, the target cell is an in vitro cell (e.g., a eukaryotic cell grown in single cell suspension or as a cell layer in vitro). In some embodiments, the target cell is an in vivo cell (e.g., a eukaryotic cell that is part of a multicellular organism). In some embodiments, the target cell is a prostate cell, e.g., a cancerous prostate cell.

The present invention further features an isolated nucleic acid comprising, in order from 5′ to 3′ and in operable linkage, a prostate cell-specific RNA polymerase II promoter, and a nucleotide sequence encoding a short interfering RNA. In some embodiments, the nucleic acid further comprises an inducible promoter 5′ of the prostate cell-specific RNA polymerase II promoter. In some embodiments, the prostate cell-specific promoter directs transcription in prostate cancer cells. In some embodiments, the prostate cell-specific promoter is a prostate-specific antigen promoter. In some embodiments, the siRNA reduces expression of a gene encoding a product that controls cell proliferation.

The present invention further features a recombinant expression vector comprising a nucleic acid that comprises, in order from 5′ to 3′ and in operable linkage, a prostate cell-specific RNA polymerase II promoter, and a nucleotide sequence encoding a short interfering RNA. The present invention further features a composition comprising the recombinant vector. The present invention further features a genetically modified host cell comprising the recombinant expression vector. In many embodiments, the genetically modified host cell is a eukaryotic cell. In some embodiments, the genetically modified host cell is an in vitro cell. In some embodiments, the genetically modified host cell is a cell of a transgenic non-human animal that comprises as a transgene the subject nucleic acid.

The present invention further features a method of reducing expression of a target gene in a prostate cell. The method generally involves introducing the recombinant expression vector (the recombinant expression vector comprising a nucleic acid that comprises, in order from 5′ to 3′ and in operable linkage, a prostate cell-specific RNA polymerase II promoter, and a nucleotide sequence encoding a short interfering RNA) into the prostate cell, where the encoded siRNA is specific for the target gene and reduces expression of the target gene. In some embodiments, the target gene is an endogenous gene. In some embodiments, the target gene encodes a product that controls cell proliferation.

### BRIEF DESCRIPTION OF THE DRAWINGS

FIGS. 1A-D depict tissue-specific silencing by expression of siRNAs from human prostate-specific antigen (PSA) promoter. The nucleotide sequence encoding GFP-specific RNAi is depicted (SEQ ID NO:2).

FIGS. 2A-E depict androgen-dependent and tissue-specific gene silencing of endogenous genes in LNCaP cells.

FIGS. 3A-C depict the biological effects of gene silencing of JNKs in LNCaP cells.

### DEFINITIONS

The term “host cell” includes an individual cell or cell culture which can be or has been a recipient of any recombinant vector(s) or synthetic polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a “recombinant host cell.” In some embodiments, a host cell is a prokaryotic cell. In other embodiments, a host cell is a eukaryotic cell.

The terms “DNA regulatory sequences,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a mammalian cell, a permanent genetic change is generally achieved by introduction of the DNA into the genome of the cell.

The term “operably linked,” as used herein, refers to a juxtaposition wherein the components so described are in a relationship permitting them to function in their intended manner. For instance, a promoter is operably linked to a coding sequence if the promoter effects transcription or expression of the coding sequence.

As used herein, the term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an episomal vector, i.e., a nucleic acid capable of extra-chromosomal replication in an appropriate host, e.g., a eukaryotic or prokaryotic host cell. Vectors capable of directing the expression of genes to which they are operatively linked are referred to herein as “expression vectors”. In the present specification, “plasmid” and “vector” are used interchangeably unless otherwise clear from the context.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from procaryotic or eukaryotic mRNA, genomic DNA sequences from prokaryotic or eukaryotic DNA, and even synthetic DNA sequences. A transcription termination sequence will usually be located 3′ to the coding sequence.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, e.g., a rodent (e.g., a rat, a mouse); an agricultural mammal (e.g., cow, a sheep, a goat, etc.); a sport mammal (e.g., a horse); a primate, e.g., a human.

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

The terms “cancer,” “neoplasm,” and “tumor” are used interchangeably herein to refer to cells which exhibit relatively autonomous growth, so that they exhibit an aberrant growth phenotype characterized by a significant loss of control of cell proliferation. Cancerous cells can be benign or malignant.

“Inhibition of gene expression” refers to the absence (or observable decrease) in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioImmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. Lower doses of administered active agent and longer times after administration of active agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

The phrase “inhibiting expression of a cellular gene by the siRNA” refers to sequence-specific inhibition of genetic expression by a small interfering RNA molecule (siRNA) characterized by degradation of specific mRNA(s). The process is also referred to as RNA interference or RNAi.

Promoters, terminators and control elements “operably linked” to a nucleic acid sequence of interest are capable of effecting the expression of the nucleic acid sequence of interest. The control elements need not be contiguous with the coding sequence, so long as they function to direct the expression thereof. Thus, for example, a promoter or terminator is “operably linked” to a coding sequence if it affects the transcription of the coding sequence. A “promoter” refers to an array of nucleic acid control sequences that direct transcription of a nucleic acid. The term “promoter” includes those promoter elements which are sufficient to render promoter-dependent gene expression controllable for cell type-specific, tissue-specific or inducible by external signals or agents. Thus, as used herein the term “promoter” is used interchangeably with the term “regulatory element(s).”

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “an siRNA” includes a plurality of such siRNAs and reference to “the cancer cell” includes reference to one or more cancer cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides nucleic acids that include a nucleotide sequence that encodes an siRNA, which nucleotide sequence is operably linked to a target cell-specific RNA polymerase II promoter. The present invention further provides vectors, including expression vectors, which include a subject nucleic acid; and host cells that harbor a subject nucleic acid or a subject expression vector. The present invention further provides methods of modulating (e.g., reducing) expression of a gene in a target cell-specific manner, the methods generally involving introducing into a cell a subject expression vector.

### Nucleic Acids, Expression Vectors, and Host Cells

The present invention provides nucleic acids that comprise a nucleotide sequence that encodes an siRNA, which nucleotide sequence is operably linked to a target cell-specific promoter RNA polymerase II promoter. The present invention further provides vectors, including expression vectors, which include a subject nucleic acid; and host cells that harbor a subject nucleic acid or a subject expression vector. Subject expression vectors are useful, when introduced into a eukaryotic cell, for modulating (e.g., reducing) gene expression in the cell in a target cell-specific manner.

A subject nucleic acid comprises a nucleotide sequence that encodes an siRNA, which nucleotide sequence is operably linked to a target cell-specific promoter RNA polymerase II promoter (“a target cell-specific RNA Pol II promoter”). Thus, a subject nucleic acid comprises, in order from 5′ to 3′ and in operable linkage, a target cell-specific RNA Pol II promoter, and a nucleotide sequence that encodes an siRNA.

A subject nucleic acid comprises an siRNA coding sequence operably linked to a tissue-specific RNA Pol II promoter. The siRNA coding sequence is typically located 3′ of the target cell-specific RNA Pol II promoter, and at a distance from the RNA Pol II promoter such that the encoded siRNA is produced in a target eukaryotic cell in which the tissue-specific promoter is functional. Typically, the siRNA coding sequence is located 3′ of the target cell-specific RNA Pol II promoter, and at a distance of from about 1 nucleotide to about 100 nucleotides 3′ of the target cell-specific RNA Pol II promoter, e.g., the siRNA coding sequence is from about 1 nucleotide to about 5 nucleotides, from about 5 nucleotides to about 10 nucleotides, from about 10 nucleotides to about 15 nucleotides, from about 15 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, from about 50 nucleotides to about 75 nucleotides, or from about 75 nucleotides to about 100 nucleotides, 3′ of the target cell-specific RNA Pol II promoter.

### Target Cell-Specific RNA Pol II Promoters

A target cell-specific RNA Pol II promoter comprises one or more regulatory elements that control transcription in a eukaryotic cell. The term “target cell-specific,” as used herein, is intended to include cell type specificity, tissue specificity, developmental stage specificity, and tumor specificity, as well as specificity for a cancerous state of a given target cell. A target cell-specific RNA Pol II promoter can be tissue-specific, tumor-specific, developmental stage-specific, cell status specific, etc., depending on the type of cell present in the target tissue or tumor. Target cell-specific RNA Pol II promoters that are suitable for use in a subject nucleic acid include, but are not limited to, cell type-specific and tissue-specific promoters, including, but not limited to, a prostate-specific antigen promoter; a hepatocyte-specific promoter; a CD4⁺ T lymphocyte-specific promoter; a glial cell-specific promoter; a neuron-specific promoter (e.g., neuron-specific enolase promoter); and the like. A target cell-specific promoter will in some embodiments include various control elements, including, but not limited to, a hypoxia-responsive element; a hormone-responsive element; an androgen-responsive element; and the like.

In some embodiments, the target cell-specific RNA Pol II promoter is an inducible promoter, e.g., the target cell-specific promoter includes one or more regulatory elements that confer inducible transcriptional control on an operably linked coding region. Inducible promoters and control elements are known in the art and include, but are not limited to, an androgen-inducible promoter; a hormone-inducible promoter; a heavy metal inducible promoter; and the like.

A target cell-specific RNA Pol II promoter is in some embodiments a “wild-type,” or “native” promoter, e.g., a naturally-occurring promoter; or has the same nucleotide sequence as a native promoter. In other embodiments, a target cell-specific RNA Pol II promoter will contain one or more differences in nucleotide sequence compared to a naturally-occurring promoter. In some embodiments, a target cell-specific RNA Pol II promoter is a synthetic promoter, e.g., the promoter is synthesized using standard recombinant and/or synthetic methods.

A target cell-specific RNA Pol II promoter is functional in a eukaryotic cell. A target cell-specific RNA Pol II promoter may comprise all or a portion of an RNA Pol II promoter from a virus, as long as the target cell-specific RNA Pol II promoter is functional in a eukaryotic cell. In some embodiments, the target cell-specific RNA Pol II promoter comprises all or a portion of a viral RNA Pol II promoter. For example, the target cell-specific RNA Pol II promoter may comprise all or a portion of a cytomegalovirus (CMV) promoter, a Human herpesvirus 1 (Herpes simplex virus type 1; see GenBank Accession No. M12474), and the like.

Cell status-specific regulatory elements include heat-inducible (i.e., heat shock) promoters, hypoxia response elements, and promoters responsive to radiation exposure, including ionizing radiation and UV radiation. For example, the promoter region of the early growth response-1 (Egr-1) gene contains an element(s) inducible by ionizing radiation. Hallahan et al. (1995) Nat. Med. 1:786-791; and Tsai-Morris et al. (1988) Nucl. Acids. Res. 16:8835-8846. Heat-inducible promoters, including heat-inducible elements, have been described. See, for example Welsh (1990) in “Stress Proteins in Biology and Medicine”, Morimoto, Tisseres, and Georgopoulos, eds. Cold Spring Harbor Laboratory Press; and Perisic et al. (1989) Cell 59:797-806. Accordingly, in some embodiments, the cell status-specific regulator element comprises an element(s) responsive to ionizing radiation. In one embodiment, this regulatory element comprises a 5′ flanking sequence of an Egr-1 gene. In other embodiments, the cell status-specific regulatory element comprises a heat shock responsive element.

Tumor cell-specific regulatory elements, and their respective target cells, include: probasin (PB), target cell, prostate cancer (PCT/US98/04132); α-fetoprotein (AFP), target cell liver cancer (PCT/US98/04084); mucin-like glycoprotein DF3 (MUCd), target cell, breast carcinoma (PCT/US98/04080); carcinoembryonic antigen (CEA), target cells, colorectal, gastric, pancreatic, breast, and lung cancers (PCT/US98/04133); plasminogen activator urokinase (uPA) and its receptor gene, target cells, breast, colon, and liver cancers (PCT/US98/04080); E2F1 (cell cycle S-phase specific promoter); target cell, tumors with disrupted retinoblastoma gene function, and HER-2/neu (c-erbB2/neu), target cell, breast, ovarian, stomach, and lung cancers (PCT/US98/04080); tyrosinase, target cell, melanoma cells as described herein and uroplakins, target cell, bladder cells.

The c-erbB2/neu gene (HER-2/neu or HER) is a transforming gene that encodes a 185 kD epidermal growth factor receptor-related transmembrane glycoprotein. In humans, the c-erbB2/neu protein is expressed during fetal development and, in adults, the protein is weakly detectable (by immunohistochemistry) in the epithelium of many normal tissues. Amplification and/or over-expression of the c-erbB2/neu gene has been associated with many human cancers, including breast, ovarian, uterine, prostate, stomach and lung cancers. The clinical consequences of overexpression of the c-erbB2/neu protein have been best studied in breast and ovarian cancer. c-erbB2/neu protein over-expression occurs in 20 to 40% of intraductal carcinomas of the breast and 30% of ovarian cancers, and is associated with a poor prognosis in subcategories of both diseases.

Human, rat and mouse c-erbB2/neu TREs have been identified and shown to confer transcriptional activity specific to c-erbB2/neu-expressing cells. Tal et al. (1987) Mol. Cell. Biol. 7:2597-2601; Hudson et al. (1990) J. Biol. Chem. 265:4389-4393; Grooteclaes et al. (1994) Cancer Res. 54:4193-4199; Ishii et al. (1987) Proc. Natl. Acad. Sci. USA 84:4374-4378; and Scott et al. (1994) J. Biol. Chem. 269:19848-19858.

The protein product of the MUC1 gene (known as mucin, MUC1 protein; episialin; polymorphic epithelial mucin or PEM; EMA; DF3 antigen; NPGP; PAS-O; or CA15.3 antigen) is normally expressed mainly at the apical surface of epithelial cells lining the glands or ducts of the stomach, pancreas, lungs, trachea, kidney, uterus, salivary glands, and mammary glands. Zotter et al. (1988) Cancer Rev. 11-12:55-101; and Girling et al. (1989) Int. J. Cancer 43:1072-1076. However, mucin is overexpressed in 75-90% of human breast carcinomas. Kufe et al. (1984) Hybridoma 3:223-232. For reviews, see Hilkens (1988) Cancer Rev. 11-12:25-54; and Taylor-Papadimitriou, et al. (1990) J. Nucl. Med. Allied Sci. 34:144-150. Mucin protein expression correlates with the degree of breast tumor differentiation. Lundy et al. (1985) Breast Cancer Res. Treat. 5:269-276.

Overexpression of the MUC1 gene in human breast carcinoma cells MCF-7 and ZR-75-1 appears to occur at the transcriptional level. Kufe et al. (1984) supra; Kovarik (1993) J. Biol. Chem. 268:9917-9926; and Abe et al. (1990) J. Cell. Physiol. 143:226-231. The regulatory sequences of the MUC1 gene have been cloned, including the approximately 0.9 kb upstream of the transcription start site which contains a TRE that appears to be involved in cell-specific transcription. Abe et al. (1993) Proc. Natl. Acad. Sci. USA 90:282-286; Kovarik et al. (1993) supra; and Kovarik et al. (1996) J. Biol. Chem. 271:18140-18147.

Carcinoembryonic Antigen (CEA) is a 180,000 Dalton, tumor-associated, glycoprotein antigen present on endodermally-derived neoplasms of the gastrointestinal tract, such as colorectal, gastric (stomach) and pancreatic cancer, as well as other adenocarcinomas such as breast and lung cancers. CEA is of clinical interest because circulating CEA can be detected in the great majority of patients with CEA-positive tumors. In lung cancer, about 50% of total cases have circulating CEA, with high concentrations of CEA (greater than 20 ng/ml) often detected in adenocarcinomas. Approximately 50% of patients with gastric carcinoma are serologically positive for CEA.

The 5′-flanking sequence of the CEA gene has been shown to confer cell-specific activity. The CEA promoter region, approximately the first 424 nucleotides upstream of the transcriptional start site in the 5′ flanking region of the gene, was shown to confer cell-specific activity by virtue of providing higher promoter activity in CEA-producing cells than in non-producing HeLa cells. Schrewe et al. (1990) Mol. Cell. Biol. 10:2738-2748. In addition, cell-specific enhancer regions have been found. See PCT/GB/02546 The CEA promoter, putative silencer, and enhancer elements appears to be contained within a region that extends approximately 14.5 kb upstream from the transcription start site. Richards et al. (1995); PCT/GB/02546. Further characterization of the 5′-flanking region of the CEA gene by Richards et al. (1995) supra indicated that two upstream regions (one between about −13.6 and about −10.7 kb, and the other between about −6.1 and about −4.0 kb), when linked to the multimerized promoter, resulted in high-level and selective expression of a reporter construct in CEA-producing LoVo and SW1463 cells. Richards et al. (1995) supra also localized the promoter region between about nt −90 and about nt +69 relative to the transcriptional start site, with the region between about nt −41 and about nt −18 being essential for expression. PCT/GB/02546 describes a series of 5′-flanking CEA fragments which confer cell-specific activity, including fragments comprising the following sequences: about nt −299 to about nt +69; about nt −90 to about nt +69; nt −14,500 to nt −10,600; nt −13,600 to nt −10,600; and nt −6100 to nt −3800, with all coordinates being relative to the transcriptional start point. In addition, cell-specific transcription activity is conferred on an operably linked gene by the CEA fragment from nt −402 to nt +69.

In some embodiments, the target cell-specific promoter is a prostate-specific promoter. In particular embodiments, the prostate-specific promoter is a prostate-specific antigen (PSA) promoter. PSA regulatory elements are described in, inter alia, U.S. Pat. Nos. 6,197,293, 5,648,478 and 5,698,443; and Lundwall et al. (1987) FEBS Lett. 214:317; Lundwall (1989) Biochim. Biophys. Res. Commun. 161:1151-1159; Riegmann et al. (1991) Molec. Endocrin. 5:1921; Schuur et al. (1996) J. Biol. Chem. 271:7043-7051; and Zhang et al. (1997) Nucleic Acids Res. 25:3143-50.

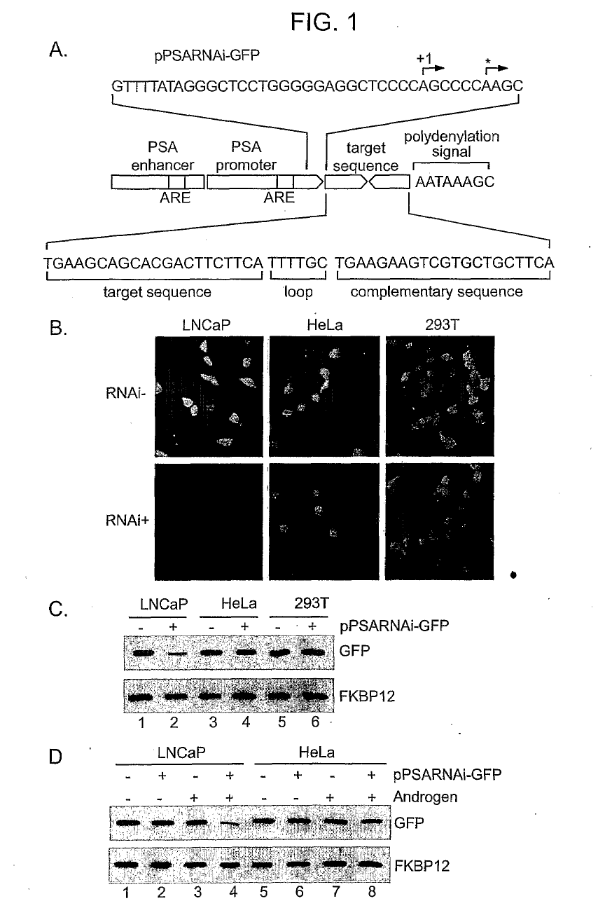

A subject nucleic acid comprises an siRNA coding sequence operably linked to a tissue-specific RNA Pot II promoter. Examples of RNA pot II promoters include, but are not limited to, housekeeping promoters, such as an actin promoter, DNA pot II promoter, PGK or a ubiquitin promoter, tissue specific promoters, for example, the albumin, globin, ovalbumin promoter sequences, skin specific promoters such as K12 or K14, inducible promoters, for example, steroid inducible promoters, tetracycline inducible promoters and the like, and viral promoters such as the SV40 early promoter, the Rous sarcoma virus (RSV) promoter and the cytomegalovirus immediate early promoter (CMV), ppol III promoter, PGK and retroviral LTR. In some embodiments, the target cell-specific RNA Pol II promoter is a prostate cell-specific RNA Pol II promoter. In some of these embodiments, the prostate cell-specific RNA Pot II promoter comprises a nucleotide sequence depicted schematically in FIG. 1A, and as set forth in SEQ ID NO:1.

siRNA-Encoding Sequences

A subject nucleic acid comprises an siRNA coding sequence operably linked to a tissue-specific RNA Pot II promoter. A subject nucleic acid comprises a nucleic acid that encodes an siRNA (also referred to herein as “an siRNA agent”). Suitable siRNA agents include siRNA agents that modulate expression of a target gene by an RNA interference mechanism. A “small interfering” or “short interfering RNA” or siRNA is a RNA duplex of nucleotides that is targeted to a gene interest (a “target gene” or a “target coding sequence”).

An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29 nucleotides (nt), 28 nt, 27 nt, 26 nt, 25 nt, 24 nt, 23 nt, 22 nt, 21 nt, 20 nt, 19 nt, 18 nt, 17 nt, 16 nt, 15 nt, 14 nt, 13 nt, 12 nt, 11 nt, or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5 nt, 6 nt, 7 nt, 8 nt, 9 nt, 10 nt, 11 nt, 12 nt, or 13 nucleotides in length. The hairpin structure can also contain 3′ or 5′ overhang portions. In some embodiments, the overhang is a 3′ or a 5′ overhang 0 nt, 1 nt, 2 nt, 3 nt, 4 nt, or 5 nucleotides in length.

In some embodiments, a subject siRNA-encoding nucleic acid comprises the nucleotide sequence depicted schematically in FIG. 1A and as set forth in SEQ ID NO:2, where the target sequence is a sequence derived from a GFP gene. In some embodiments, the encoded siRNA comprises the nucleotide sequence set forth in SEQ ID NO:3. In some embodiments, a subject siRNA-encoding nucleic acid comprises a PSA enhancer and promoter sequence, as set forth in SEQ ID NO: 1; and a target sequence derived from a JNK gene. In some of these embodiments, the JNK target sequence is 5′-GATCAGTGGAATAAAGTTATTTTTGCAATAACTTTATTCCACTGATC-3′ (SEQ ID NO:4), where the loop nucleotides are in bold text, and the complementary sequence is underlined. In some embodiments, a subject siRNA-encoding nucleic acid comprises a PSA enhancer and promoter sequence, as set forth in SEQ ID NO: 1; and a target sequence derived from a PI3K gene. In some of these embodiments, the PI3K target sequence is 5′-AAGCAAGTTCACAATTACCCATTTGCTGGGTAATTGTGAACTTGCTT-3′ (SEQ ID NO:5), where the loop nucleotides are in bold text, and the complementary sequence is underlined. Given the guidance provided in the instant specification, those skilled in the art can readily generate other siRNA-encoding nucleic acids comprising a tissue-specific RNA Pol II promoter operably linked to a nucleotide sequence encoding an siRNA comprising a target sequence that functions to reduce expression of any of a wide variety of target genes.

### Preparing a Subject Nucleic Acid

Preparation of a Subject Nucleic Acid Accomplished Utilizing any of the Methods Known to one skilled in the art. Changes in nucleotide sequence of any given nucleic acid is accomplished by any of various standard methods, including site-specific mutagenesis, polymerase chain reaction (PCR) amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Using these techniques, it is possible to insert or delete, at will, a polynucleotide of any length into a subject nucleic acid.

A subject nucleic acid, or a fragment of a subject nucleic acid, will in some embodiments be prepared using chemical synthesis of linear oligonucleotides which may be carried out utilizing techniques well known in the art. The synthesis method selected will depend on various factors including the length of the desired nucleic acid and such choice is within the skill of the ordinary artisan. Oligonucleotides are typically synthesized chemically according to the solid phase phosphoramidite triester method described by Beaucage and Caruthers, Tetrahedron Letts., 22(20):1859-1862 (1981), e.g., using an automated synthesizer, as described in Needham-VanDevanter et al., Nucleic Acids Res., 12:6159-6168 (1984). Oligonucleotides can also be custom made and ordered from a variety of commercial sources known to persons of skill in the art.

Synthetic linear oligonucleotides maybe purified by polyacrylamide gel electrophoresis, or by any of a number of chromatographic methods, including gel chromatography and high pressure liquid chromatography. The sequence of the synthetic oligonucleotides can be verified using the chemical degradation method of Maxam and Gilbert in Grossman and Moldave (eds.) Academic Press, New York, Methods in Enzymology, 65:499-560 (1980). If modified bases are incorporated into the oligonucleotide, and particularly if modified phosphodiester linkages are used, then the synthetic procedures are altered as needed according to known procedures. In this regard, Uhlmann, et al., Chemical Reviews, 90:543-584 (1990) provide references and outline procedures for making oligonucleotides with modified bases and modified phosphodiester linkages. Sequences of short oligonucleotides can also be analyzed by laser desorption mass spectroscopy or by fast atom bombardment (McNeal, et al., J. Am. Chem. Soc., 104:976 (1982); Viari, et al., Biomed. Enciron. Mass Spectrom., 14:83 (1987); Grotjahn et al., Nuc. Acid Res., 10:4671 (1982)).

Linear oligonucleotides may also be prepared by polymerase chain reaction (PCR) techniques as described, for example, by Saiki et al., Science, 239:487 (1988). In vitro amplification techniques suitable for amplifying nucleotide sequences are also well known in the art. Examples of such techniques including the polymerase chain reaction (PCR), the ligase chain reaction (LCR), Qβ-replicase amplification and other RNA polymerase mediated techniques (e.g., NASBA) are found in Berger, Sambrook, and Ausubel, as well as Mullis et al., (1987) U.S. Pat. No. 4,683,202; PCR Protocols A Guide to Methods and Applications (Innis et al., eds) Academic Press Inc., San Diego, Calif. (1990) (Innis); Arnheim & Levinson (Oct. 1, 1990) C&EN 36-47; The Journal Of NIH Research, 3:81-94 (1991); (Kwoh et al., (1989) Proc. Natl. Acad. Sci. USA, 86:1173; Guatelli et al., Proc. Natl. Acad. Sci. USA, 87:1874 (1990); Lomell et al., J. Clin. Chem., 35:1826 (1989); Landegren et al., Science, 241:1077-1080 (1988); Van Brunt, Biotechnology, 8:291-294 (1990); Wu and Wallace, Gene, 4:560 (1989); Barringer et al., Gene, 89:117 (1990), and Sooknanan and Malek, Biotechnology, 13:563-564 (1995). Improved methods of cloning in vitro amplified nucleic acids are described in Wallace et al., U.S. Pat. No. 5,426,039.

### Recombinant Vectors

The above nucleic acid constructs comprising an siRNA coding domain operably linked to a target cell-specific RNA Pol II promoter are, in many embodiments, present in a vector. A vector that comprises a subject nucleic acid is referred to herein as a “recombinant vector.” The constructs may be present on any convenient type of vector, where representative vectors of interest include, but are not limited to: plasmid vectors, viral vectors, and the like.

Certain types of vectors allow the expression cassettes of the present invention to be amplified. Other types of vectors are necessary for efficient introduction of subject nucleic acid to cells and their stable expression once introduced. Any vector capable of accepting a subject nucleic acid is contemplated as a suitable recombinant vector for the purposes of the invention. The vector may be any circular or linear length of DNA that either integrates into the host genome or is maintained in episomal form. Vectors may require additional manipulation or particular conditions to be efficiently incorporated into a host cell (e.g., many expression plasmids), or can be part of a self-integrating, cell specific system (e.g., a recombinant virus). The vector is in some embodiments functional in a prokaryotic cell, where such vectors function to propagate the recombinant vector. The vector is in some embodiments functional in a eukaryotic cell, where the vector will in many embodiments be an expression vector.

Representative eukaryotic plasmid vectors of interest include, for example: pCMVneo, pShuttle, pDNR and Ad-X (Clontech Laboratories, Inc.); as well as BPV, EBV, vaccinia, SV40, 2-micron circle, pcDNA3.1, pcDNA3.1/GS, pYES2/GS, pMT, p IND, pIND(Spl), pVgRXR, and the like, or their derivatives. Such plasmids are well known in the art (Botstein et al., Miami Wntr. SyTnp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Dilon et at., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980.

Certain vectors, “expression vectors”, are capable of directing the expression of genes. Any expression vector comprising an expression cassette of the present invention qualifies as an expression cassette of the present invention. In general, expression vectors of utility in recombinant DNA techniques often are in the form of plasmids. However, preferred vector systems of the present invention are viral vectors, e.g., replication defective retroviruses, lentiviruses, adenoviruses; adeno-associated viruses (e.g., AAV-1, AAV-2, etc.; baculovirus, CaMV; herpesviruses; vaccinia virus; and the like.

Examples of suitable prokaryotic expression vectors that can be engineered to accept a subject nucleic acid include pTrc (Amann et al., Gene, 69:301-315 (1988)) and pBluescript (Stratagene, San Diego, Calif.). Examples of vectors for expression in yeast S. cerevisiae include pYepSec1 (Baldari et al., EMBO J., 6:229-234 (1987)), pMFa (Kurjan and Herskowitz, Cell, 30:933-943 (1982)), pJRY88 (Schultz et al., Gene, 54:113-123 (1987)), pYES2 (Invitrogen, Carlsbad, Calif.), and pPicZ (Invitrogen, Carlsbad, Calif.). Baculovirus vectors are often used for expression of dsRNAs in cultured insect cells (e.g., Sf9 cells see, U.S. Pat. No. 4,745,051) and include the pAc series (Smith et al., Mol. Cell. Biol., 3:2156-2165 (1983)), the pVL series (Lucklow and Summers, Virology, 170:31-39 (1989)) and pBlueBac (available from Invitrogen, San Diego).

Infection of cells with a viral vector will in some embodiments be used for introducing expression cassettes of the present invention into cells. The viral vector approach has the advantage that a large proportion of cells receive the expression cassette, which can obviate the need for selection of cells that have been successfully transfected. Exemplary mammalian viral vector systems include retroviral vectors, lentiviral vectors, adenoviral vectors, adeno-associated type 1 (“AAV-1”) or adeno-associated type 2 (“AAV-2”) vectors, hepatitis delta vectors, live, attenuated delta viruses, and herpes viral vectors.

In some embodiments, a subject recombinant vector is a retroviral vector. Retroviruses are RNA viruses that are useful for stably incorporating genetic information into the host cell genome. When a retrovirus infects cells, their RNA genomes are converted to a dsDNA form (by the viral enzyme reverse transcriptase). The viral DNA is efficiently integrated into the host genome, where it permanently resides, replicating along with host DNA at each cell division. The integrated provirus steadily produces viral RNA from a strong promoter located at the end of the genome (in a sequence called the long terminal repeat or LTR). This viral RNA serves both as mRNA for the production of viral proteins and as genomic RNA for new viruses. Viruses are assembled in the cytoplasm and bud from the cell membrane, usually with little effect on the cell's health. Thus, the retrovirus genome becomes a permanent part of the host cell genome, and any foreign gene placed in a retrovirus ought to be expressed in the cells indefinitely. Retroviruses are therefore attractive vectors because they can permanently express a foreign gene in cells. Most or possibly all regions of the host genome are accessible to retroviral integration (Withers-Ward et al., Genes Dev., 8:1473-1487 (1994)). Moreover, they can infect virtually every type of mammalian cell, making them exceptionally versatile.

Retroviral vector particles are prepared by recombinantly inserting a subject nucleic acid into a retroviral vector and packaging the vector with retroviral proteins by use of a packaging cell line or by co-transfecting non-packaging cell lines with the retroviral vector and additional vectors that express retroviral proteins. The resultant retroviral vector particle is generally incapable of replication in the host cell and is capable of integrating into the host cell genome as a proviral sequence containing the expression cassette containing a nucleic acid encoding an siRNA. As a result, the host cell produces the siRNA encoded by the subject recombinant expression vector.

Packaging cell lines are generally used to prepare the retroviral vector particles. A packaging cell line is a genetically constructed mammalian tissue culture cell line that produces the necessary viral structural proteins required for packaging, but which is incapable of producing infectious virions. Retroviral vectors, on the other hand, lack the structural genes but have the nucleic acid sequences necessary for packaging. To prepare a packaging cell line, an infectious clone of a desired retrovirus, in which the packaging site has been deleted, is constructed. Cells comprising this construct will express all structural proteins but the introduced DNA will be incapable of being packaged. Alternatively, packaging cell lines can be produced by introducing into a cell line one or more expression plasmids encoding the appropriate core and envelope proteins. In these cells, the gag, pol, and env genes can be derived from the same or different retroviruses.

A number of packaging cell lines suitable for the present invention are available in the art. Examples of these cell lines include Crip, GPE86, PA317 and PG13. See, e.g., Miller et al., J. Virol., 65:2220-2224 (1991). Examples of other packaging cell lines are described in Cone and Mulligan, Proceedings of the National Academy of Sciences, U.S.A., 81:6349-6353 (1984) and in Danos and Mulligan, Proceedings of the National Academy of Sciences, U.S.A., 85:6460-6464 (1988); Eglitis et al., Biotechniques, 6:608-614 (1988); Miller et al., Biotechniques, 7:981-990 (1989). Amphotropic or xenotropic envelope proteins, such as those produced by PA317 and GPX packaging cell lines may also be used to package the retroviral vectors.

Defective retroviruses are well characterized for use in gene transfer to mammalian cells (for a review see Miller, A. D., Blood, 76:271 (1990)). A recombinant retrovirus can be constructed having a subject nucleic acid inserted into the retroviral genome. Additionally, portions of the retroviral genome can be removed to render the retrovirus replication defective. The replication defective retrovirus is then packaged into virions that can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals.

Examples of suitable retroviruses include pLJ, pZIP, pWE and pEM which are well known to those skilled in the art. Examples of suitable packaging virus lines include ψCrip, ψCre, ψ2, and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al., Science, 230:1395-1398 (1985); Danos and Mulligan, Proc. Natl. Acad. Sci. USA, 85:6460-6464 (1988); Wilson et al., Proc. Natl. Acad. Sci. USA, 85:3014-3018 (1988); Armentano et al., Proc. Natl. Acad. Sci. USA, 87:6141-6145 (1990); Huber et al., Proc. Natl. Acad. Sci. USA, 88:8039-8043 (1991); Ferry et al., Proc. Natl. Acad. Sci. USA, 88:8377-8381 (1991); Chowdhury et al., Science, 254:1802-1805 (1991); van Beusechem et al., Proc. Natl. Acad. Sci. USA, 89:7640-7644 (1992); Kay et al., Human Gene Therapy, 3:641-647 (1992); Dai et al., Proc. Natl. Acad. Sci. USA, 89:10892-10895 (1992); Hwu et al., J. Immunol., 150:4:104-115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573; EPA 0 178 220; U.S. Pat. No. 4,405,712; Gilboa, Biotechniques, 4:504-512 (1986); Mann et al., Cell, 33:153-159 (1983); Cone and Mulligan, Proc. Natl. Acad. Sci. USA, 81:6349-6353 (1984); Eglitis et al., Biotechniques 6:608-614 (1988); Miller et al., Biotechniques, 7:981-990 (1989); Miller, Nature (1992), supra; Mulligan, Science, 260:926-932 (1993); and Gould et al., and International Patent Application No. WO 92/07943 entitled “Retroviral Vectors Useful in Gene Therapy.”).

The genome of an adenovirus can be manipulated such that it includes a subject nucleic acid, but is inactivated in terms of its ability to replicate in a normal lytic viral life cycle. See for example Berkner et al., BioTechniques, 6:616 (1988); Rosenfeld et al., Science, 252:431-434 (1991); and Rosenfeld et al., Cell, 68:143-155 (1992). Suitable adenoviral vectors derived from the adenovirus strain Ad type 5 dl 324 or other strains of adenovirus (e.g., Adz, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al., Proc. Natl. Acad. Sci. USA, 89):6482-6486 (1992)), hepatocytes (Herz and Gerard, Proc. Natl. Acad. Sci. USA, 90:2812-2816 (1993)) and muscle cells (Quantin et al., Proc. Natl. Acad. Sci. USA, 89:2581-2584 (1992)).

Adeno-associated virus (AAV) is a naturally occurring defective virus that requires another virus, such as an adenovirus or a herpes virus, as a helper virus for efficient replication and a productive life cycle. (For a review see Muzyczka et al., Curr. Topics in Micro. and Immunol., 158:97-129 (1992)). It exhibits a high frequency of stable integration (see for example Flotte et al., Am. J. Respir. Cell. Mol. Biol., 7:349-356 (1992); Samulski et al., J. Virol., 63:3822-3828 (1989); and McLaughlin et al., J. Virol, 62:1963-1973 (1989); Flotte, et al., Gene Ther., 2:29-37 (1995); Zeitlin, et al., Gene Ther., 2:623-31 (1995); Baudard, et al., Hum. Gene Ther., 7:1309-22 (1996)). Vectors containing as little as 300 base pairs of AAV can be packaged and can integrate. Space for exogenous nucleic acid is limited to about 4.5 kb, well in excess of the overall size of the expression vectors of the invention. An AAV vector, such as that described in Tratschin et al., Mol. Cell. Biol., 5:3251-3260 (1985) can be used to introduce the expression vector into cells. A variety of nucleic acids have been introduced into different cell types using AAV vectors (see for example Hermonat et al., Proc. Natl. Acad. Sci. USA, 81:6466-6470 (1984); Tratschin et al., Mol. Cell. Biol., 4:2072-2081 (1985); Wondisford et al., Mol. Endocrinol., 2:32-39 (1988); Tratschin et al., J. Virol., 51:611-619 (1984); and Flotte et al., J. Biol. Chem., 268:3781-3790 (1993)).

A subject nucleic acid will in some embodiments be incorporated into lentiviral vectors. In this regard, see:_Qin et al. (2003) Proc. Natl. Acad. Sci. USA 100: 183-188; Miyoshi et al. (1998) J. Virol. 72: 8150-8157; Tisconia et al. (2003) Proc. Natl. Acad. Sci. USA 100: 1844-1848; and Pfeifer et al. (2002) Proc. Natl. Acad. Sci. USA 99: 2140-2145. Lentiviral vector kits are available from Invitrogen (Carlsbad, Calif.).

A subject recombinant vector will in some embodiments include one or more selectable markers. A number of selection systems may be used, including but not limited to the herpes simplex virus thymidine kinase (Wigler, et al., Cell, 11:223 (1977)), hypoxanthine-guanine phosphoribosyltransferase (Szybalska & Szybalski, Proc. Natl. Acad. Sci. USA, 48:2026 (1962)), and adenine phosphoribosyltransferase (Lowy et al., Cell, 22:817 (1980)) genes can be employed in tk⁻, hgprt⁻ or aprt⁻ cells, respectively. Also, antimetabolite resistance can be used as the basis of selection for dhfr, which confers resistance to methotrexate (Wigler et al., Natl. Acad. Sci. USA, 77:3567 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418 (Colberre-Garapin et al., J. Mol. Biol., 150:1 (1981)); and hygro, which confers resistance to hygromycin (Santerre, et al., Gene, 30:147 (1984)). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047 (1988)); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.).

### Host Cells

The present invention provides host cells, e.g., genetically modified host cells, which comprise a subject nucleic acid and/or a subject recombinant vector. A subject recombinant vector can be used to transform (“genetically modify”) any eukaryotic or prokaryotic cell for a variety of purposes including, but not limited to, amplification of the recombinant vector, and modulation of gene expression. Eukaryotic cell types that can serve as targets for vectors containing expression cassettes of the present invention include primary cell cultures, cell lines, yeast, and cellular populations in whole organs and organisms. A genetically modified host cell is in some embodiments a cell in vitro (e.g., an “isolated” genetically modified host cell), and in other embodiments a genetically modified host cell is a cell in vivo. In vivo genetically modified host cells include cells that are part of tissues or organs; tumor cells; prostate cells; CD4⁺ T cells; etc.

Suitable eukaryotic host cells include, but are not limited to, monkey kidney CVI line transformed by SV40 (COS-7, ATCC CRL 1651); human embryonic kidney line (293, Graham et al., J. Gen Virol., 36:59 (1977)); baby hamster kidney cells (BHK, ATCC CCL 10); Chinese hamster ovary-cells-DHFR (CHO, Urlaub and Chasin, Proc. Natl. Acad. Sci. (USA), 77:4216 (1980)); mouse sertoli cells (TM4, Mather, Biol. Reprod., 23:243-251 (1980)); monkey kidney cells (CVI ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (WI 38, ATCC CCL 75); human liver cells (hep G2, HB 8065); mouse mammary tumor (MMT 060562, ATCC CCL51); TRI cells (Mather et al., Annals N.Y. Acad. Sci, 383:44-68 (1982)); human B cells (Daudi, ATCC CCL 213); human T cells (MOLT-4, ATCC CRL 1582); and human macrophage cells (U-937, ATCC CRL 1593). The cells can be maintained according to standard methods well known to those of skill in the art (see, e.g., Freshney, Culture of Animal Cells, A Manual of Basic Technique, (3d ed.) Wiley-Liss, New York (1994); Kuchler et al., Biochemical Methods in Cell Culture and Virology (1977), Kuchler, R. J., Dowden, Hutchinson and Ross, Inc. and the references cited therein). Cultured cell systems often will be in the form of monolayers of cells, although cell suspensions are also used.

### Introducing a Recombinant Vector into a Host Cell

A subject recombinant vector may be introduced into a host cell utilizing a vehicle, or by various physical methods. Representative examples of such methods include transformation using calcium phosphate precipitation (Dubensky et al., PNAS, 81:7529-7533 (1984)), direct microinjection of such nucleic acid molecules into intact target cells (Acsadi et al., Nature, 352:815-818 (1991)), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS, 89:6094 (1990)), lipofection (Felgner et al., Proc. Natl. Acad. Sci. USA, 84:7413-7417 (1989)), microprojectile bombardment (Williams et al., PNAS, 88:2726-2730 (1991)), polycation compounds such as polylysine, receptor specific ligands, liposomes entrapping the nucleic acid molecules, and spheroplast fusion whereby E. coli containing the nucleic acid molecules are stripped of their outer cell walls and fused to animal cells using polyethylene glycol.

### Methods of Reducing Gene Expression in a Target Cell-Specific Manner

The present invention provides methods of reducing expression of a target gene or coding sequence in a eukaryotic cell in a target cell-specific manner. The methods generally involve introducing a subject recombinant expression vector into a eukaryotic cell, such that the siRNA encoded by the vector is produced in the cell, and the siRNA inhibits expression of a target gene or coding sequence. In some embodiments the eukaryotic cell is in vitro. In other embodiments, the eukaryotic cell is in vivo. In some embodiments, the eukaryotic cell is an in vitro eukaryotic host cell that is grown as a unicellular entity in in vitro cell culture. In other embodiments, the eukaryotic cell is an in vitro eukaryotic cell that is grown as a monolayer in in vitro cell culture. In other embodiments, the eukaryotic cell is a totipotent or pluripotent cell. In other embodiments, the eukaryotic cell is a stem cell, a progenitor cell, or a progeny thereof. In other embodiments, the eukaryotic cell is part of a multicellular organism.

By reducing expression is meant that the level of expression of a target gene or coding sequence is reduced or inhibited by at least about 10%, at least about 20%, at least about 25%, at least about 30%, at least about 40%, at least about 50%, at least about 60%, at least about 70%, at least about 80%, at least about 90%, or more, or more, as compared to a control. In certain embodiments, the expression of the target gene is reduced to such an extent that expression of the target gene/coding sequence is effectively inhibited, such that expression is undetectable. By modulating expression of a target gene is meant altering, e.g., reducing, transcription/translation of a coding sequence, e.g., genomic DNA, mRNA etc., into a polypeptide, e.g., protein, product.

The siRNA agent can be introduced into a host cell using any convenient protocol, where the protocol employed is typically a nucleic acid administration protocol, where a number of different such protocols are known in the art. The following discussion provides a review of representative nucleic acid administration protocols that may be employed. The nucleic acids may be introduced into tissues or host cells by any number of routes, including viral infection, microinjection, or fusion of vesicles. Jet injection may also be used for intra-muscular administration, as described by Furth et al. (1992), Anal Biochem 205:365-368. The nucleic acids may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells.

Expression vectors may be used to introduce the nucleic acids into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

A subject nucleic acid can be fed directly to, injected into, the host organism containing the target gene. The siRNA agent may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution. The agent may be introduced in an amount which allows delivery of at least one copy per cell. Higher doses (e.g., at least 5, 10, 100, 500 or 1000 copies per cell) of the agent may yield more effective inhibition; lower doses may also be useful for specific applications.

Additional nucleic acid delivery protocols of interest include, but are not limited to: those described in, e.g., U.S. Pat. Nos. 5,985,847 and 5,922,687; WO/11092; Acsadi et al., New Biol. (1991) 3:71-81; Hickman et al., Hum. Gen. Ther. (1994) 5:1477-1483; and Wolff et al., Science (1990) 247: 1465-1468; etc.

A subject nucleic acid or a subject recombinant vector, is also referred to herein as an “siRNA agent” or an “active agent.” Depending on the nature of the siRNA agent (the “active agent”), the active agent(s) may be administered to the host using any convenient means capable of resulting in the desired modulation of target gene expression. Thus, the agent can be incorporated into a variety of formulations for therapeutic administration. More particularly, the siRNA agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intramuscular, intratumoral, subcutaneous, intraocular, intradermal, transdermal, intracheal, etc., administration.

In pharmaceutical dosage forms, the agents may be administered alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means.

### Research Applications

A subject nucleic acid finds utility in a variety of research applications. An inducible Pol II-mediated expression vector, as described herein, is useful for controlling the expression of siRNA or short hairpin RNA (shRNA) for functional analysis of any target gene. For example, an inducible Pol II-mediated expression vector, as described herein, is useful for controlling the expression of siRNA or shRNA for functional analysis of cell viability-essential genes. This approach is a cost- and time-effective method to study the function(s) of a targeted gene in cell-based systems and transgenic animals.

In some embodiments, a subject nucleic acid is introduced into a eukaryotic cell in vitro, and the effect, if any, of the nucleic acid on expression of a target gene is determined. Examples of methods include methods to determine the level of mRNA encoded by the target gene (e.g., Northern hybridization analysis, reverse transcription-PCR analysis, and the like); immunological methods to determine the level of protein encoded by the target gene (e.g., immunological methods such as enzyme-linked immunosorbent assay, radioimmunoassay, and the like; methods to determine the level of activity of protein encoded by the target gene (e.g., enzymatic assays; assays for receptor function; assays for activity in regulating cell cycle (e.g., cell proliferation assays, assays to measure apoptosis, etc.); and the like). In some embodiments, a subject nucleic acid is introduced into a eukaryotic cell in vitro, and the effect, if any, of the nucleic acid on viability of the cell is determined. In some embodiments, a subject nucleic acid is introduced into a eukaryotic cell in vitro, and the effect, if any, of the nucleic acid on proliferation of the cell is determined. In some embodiments, a subject nucleic acid is introduced into a eukaryotic cell in vitro, and the effect, if any, of the nucleic acid on apoptosis is determined.

In some embodiments, a subject nucleic acid is used as a transgene to generate a transgenic non-human animal. Numerous publications describe methods of making transgenic non-human animals. See, e.g., Transgenesis Techniques: Principles and Protocols D. Murphy and D. A. Carter, ed. (June 1993) Humana Press; Transgenic Animal Technology: A Laboratory Handbook C. A. Pinkert, ed. (January 1994) Academic Press; Transgenic Animals F. Grosveld and G Kollias, eds. (July 1992) Academic Press; Embryonal Stem Cells: Introducing Planned Changes into the Animal Germline M. L. Hooper (January 1993) Gordon & Breach Science Pub; and Transgenic Animal Technology: A Laboratory Handbook, 2^{nd} edition, C. A. Pinker (November 2002) Elsevier Science.

Typically, a transgene comprising a subject nucleic acid is introduced into a pluripotent or totipotent cell such that the transgene is integrated into the genome of the cell, and transferring the cell into an oviduct of a synchronized recipient female of the same species as the cell. Transgenic animals comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. A transgenic animal may be heterozygous or homozygous for the transgene. During the initial construction of the animal, “chimeras” or “chimeric animals” are generated in some methods (e.g., where ES cells are used), in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

In some embodiments, the transgene is introduced into a somatic cell, where the transgene is integrated into the genome, forming a genetically modified somatic cell, and the nucleus of the genetically modified somatic cell is transferred into a single-cell embryo, forming a genetically modified embryo. The genetically modified single-cell embryo is then transferred into an oviduct of a recipient female, and the embryo is allowed to develop into a mature transgenic animal.

Transgenic animals also can be generated using methods of nuclear transfer or cloning using embryonic or adult cell lines as described for example in Campbell et al. (1996) Nature 380: 64-66; and Wilmut et al. (1997) Nature 385: 810-813. Cytoplasmic injection of DNA can be used, as described in U.S. Pat. No. 5,523,222.

Transgenic animals are analyzed to determine the effect, if any, of a subject nucleic acid on expression of a target gene. The method used to determine the effect of a subject nucleic acid on expression of a target gene will depend, in part, on the target gene. Examples of methods include methods to determine the level of mRNA encoded by the target gene (e.g., Northern hybridization analysis, reverse transcription-PCR analysis, and the like) immunological methods to determine the level of protein encoded by the target gene (e.g., immunological methods such as enzyme-linked immunosorbent assay, radioimmunoassay, and the like; methods to determine the level of activity of protein encoded by the target gene (e.g., enzymatic assays, assays for receptor function, and the like). In some embodiments, the transgenic animal is used to assess the effect of a subject nucleic acid on reducing tumor growth, e.g., in prostate cancer cells. Prostate cancer animal models have been described in the art; and any known prostate cancer animal model can be used. See, e.g., Wang, S. et al. (2003) Cancer Cell. 2003 September; 4(3):209-211, describing a Pten null animal model; Garabedian et al. (1998) Proc. Natl. Acad. Sci. USA 95:15382; Winter et al. (2003) Prostate Cancer Prostatic Disease 6:204-211; U.S. Pat. No. 5,917,124.

### Treatment Methods

The present invention provides methods of treating various disorders, the methods generally involving administering a subject expression vector to an individual, such that the expression vector enters a cell of the individual, the siRNA encoded by the expression vector is produced in a target cell-specific manner, and the siRNA reduces expression of the target gene or coding sequence in the cell. The subject siRNA encoding nucleic acids or siRNA products thereof also find use in a variety of therapeutic applications in which it is desired to selectively modulate, e.g., one or more target genes in a host, e.g., whole mammal, or portion thereof, e.g., tissue, organ, etc, as well as in cells present therein. In such methods, an effective amount of the subject siRNA encoding molecules or siRNA products thereof is administered to the host or target portion thereof. By effective amount is meant a dosage sufficient to selectively modulate expression of the target gene(s), as desired. As indicated above, in many embodiments of this type of application, the subject methods are employed to reduce/inhibit expression of one or more target genes in the host or portion thereof in order to achieve a desired therapeutic outcome.

Depending on the nature of the condition being treated, the target gene may be a gene derived from the cell, an endogenous gene, a pathologically mutated gene, e.g. a cancer causing gene, one or more genes whose expression causes or is related to heart disease, lung disease, Alzheimer's disease, Parkinson's disease, diabetes, arthritis, etc.; a transgene, or a gene of a pathogen which is present in the cell after infection thereof, e.g., a viral (e.g., HIV-Human Immunodeficiency Virus; HBV-Hepatitis B virus; HCV-Hepatitis C virus; Herpes-simplex 1 and 2; Varicella Zoster (Chicken pox and Shingles); Rhinovirus (common cold and flu); any other viral form) or bacterial pathogen. Depending on the particular target gene and the dose of construct or siRNA product delivered, the procedure may provide partial or complete loss of function for the target gene. Lower doses of injected material and longer times after administration of siRNA may result in inhibition in a smaller fraction of cells.

The present invention is not limited to modulation of expression of any specific type of target gene or nucleotide sequence. Target genes include any gene encoding a target gene product (RNA or protein) that is deleterious (e.g., pathological); a target gene product that is malfunctioning; a target gene product. Target gene products include, but are not limited to, huntingtin; hepatitis C virus; human immunodeficiency virus; amyloid precursor protein; tau; a protein that includes a polyglutamine repeat; a herpes virus (e.g., varicella zoster); any pathological virus; and the like. Representative classes of target genes of interest include but are not limited to: developmental genes (e.g., adhesion molecules, cyclin kinase inhibitors, cell-cycle control genes, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA 1, BRCA2, MADH4, MCC, NF 1, NF2, RB 1, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nucleolar RNAs, RNA transport factors), translation factors, telomerase reverse transcriptase); etc.

As such a subject recombinant vector that includes a nucleic acid encoding an siRNA is useful for treating a variety of disorders and conditions, including, but not limited to, neurodegenerative diseases, e.g., a trinucleotide-repeat disease, such as a disease associated with polyglutamine repeats, e.g., Huntington's disease, spinocerebellar ataxia, spinal and bulbar muscular atrophy (SBMA), dentatorubropallidoluysian atrophy (DRPLA), etc.; an acquired pathology (e.g., a disease or syndrome manifested by an abnormal physiological, biochemical, cellular, structural, or molecular biological state) such as a viral infection, e.g., hepatitis that occurs or may occur as a result of an HCV infection, acquired immunodeficiency syndrome, which occurs as a result of an HIV infection; cancer; and the like.

### Cancer Treatment

As one non-limiting example, a gene involved in cell proliferation is the target gene. A subject recombinant expression vector that comprises an siRNA-encoding nucleotide sequence operably linked to a promoter that directs expression in a particular type of cancer cell is administered to an individual. The encoded siRNA targets a gene involved in cell proliferation, e.g., a cyclin-dependent kinase. The siRNA reduces expression of the gene involved in cell proliferation, and, as a result, proliferation of the target cell is reduced. A gene involved in cell proliferation is a gene whose product (e.g., RNA or protein) controls cell proliferation.

In some embodiments, a subject recombinant vector reduces proliferation of a target cell that is genetically modified with the subject recombinant vector by at least about 10%, at least about 20%, at least about 30%, at least about 40%, at least about 50%, at least about 60%, at least about 70%, or at least about 80%, or more, compared to the proliferation of the target cell not genetically modified with the subject recombinant vector.

In some embodiments, a subject recombinant vector, when administered into an individual having a tumor, is effective to reduce the tumor load in the individual by at least about 10%, at least about 20%, at least about 30%, at least about 40%, at least about 50%, at least about 60%, at least about 70%, or at least about 80%, or more, compared to the tumor load in the untreated individual.

In some embodiments, a subject recombinant vector induces apoptosis in a cell that is genetically modified with the subject recombinant vector. Apoptosis can be assayed using any known method. Assays can be conducted on cell populations or an individual cell, and include morphological assays and biochemical assays. A non-limiting example of a method of determining the level of apoptosis in a cell population is TUNEL (TdT-mediated dUTP nick-end labeling) labeling of the 3′-OH free end of DNA fragments produced during apoptosis (Gavrieli et al. (1992) J. Cell Biol. 119:493). The TUNEL method consists of catalytically adding a nucleotide, which has been conjugated to a chromogen system or to a fluorescent tag, to the 3′-OH end of the 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus). Another marker that is currently available is annexin, sold under the trademark APOPTEST™. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. Other methods of testing for apoptosis are known in the art and can be used, including, e.g., the method disclosed in U.S. Pat. No. 6,048,703.

A subject nucleic acid, when introduced into a tumor cell, is effective to reduce the growth rate of the tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, an “effective amount” of a subject siRNA is an amount that is sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be a genetically identical animal not treated with the siRNA. In non-experimental systems, a suitable control may be the tumor load present before administering the siRNA. Other suitable controls may be a placebo control.

Whether growth of a tumor is inhibited can be determined using any known method, including, but not limited to, a proliferation assay as described in the Example; a 3H-thymidine uptake assay; and the like.

The methods are useful for treating a wide variety of cancers, including carcinomas, sarcomas, leukemias, and lymphomas. In particular embodiments of interest, the present invention provides methods of reducing prostate cancer growth.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

### Combination Therapies

In some embodiments, a subject siRNA is administered as an adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, allyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlonnethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC-609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triarncinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressag) (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL®, TAXOTERE® (a formulation of docetaxel), 10-desacetyl analogs of paclitaxel and 3 N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere® docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) colony-stimulating factors; and (7) inhibitors of angiogenesis.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Construction and Characterization of Constructs Comprising siRNA Coding Sequences Under Transcriptional Control of Tissue-Specific RNA Pol II Promoters

### Materials and Methods

### Plasmids

Sequences of fragments of the human PSA enhancer, the PSA promoter, the target sequence for green fluorescent protein (GFP), and the polyadenylation signal (AATAAA) were obtained by polymerase chain reaction (PCR) amplification, using pPSAR2.4K-PCPSA-P-Lux as a template; Pang et al. (1997) Cancer Res. 57:495-499. For cloning purposes, forward primer 5′-ATCTCGAGCCGAGAAATTAATTGTGGCG-3′ (SEQ ID NO:6) is flanked with an XhoI site at the 5′ end (underlined); reverse primer 5′-ATGAATTCTTTATTAAGCTTGAAGCAGCACGACTTCTTCAGCAAAATGAAGAAGT CGTGCTGCTTCAGCTTGGGGCTGGGGAGCCTCC-3′ (PSA-GFP; SEQ ID NO:7); reverse primer 5′-ATGAATTCTTTATTGATCAGTGGAATAAAGTTATTCGAAAATAACTTTATTTTATT CCACTGATCGCTTGGGGCTGGGGAGCCTCC-3′ [PSA-c-Jun-HN₂-terminal kinase (JNK); SEQ ID NO:8]; and reverse primer 5′-ATGAATTCTTTATTAAGCAAGTTCACAATTACCCACGAATGGGTAATTGTGAACTT GCTTGCTTGGGGCTGGGGAGCCTCC-3′ [PSA-phosphatidylinositol 3-kinase (PI3K); SEQ ID NO:9] were used. The reverse primers are flanked with EcoRI sites (underlined) and a synthetic polyadenylation sequence (in bold) at the 5′ end. PCR products were digested with XhoI and EcoRI and then subcloned into pBluescript II KS+ (Invitrogen, Carlsbad, Calif., USA) to generate pPSARNAi-GFP.

A lentiviral vector was used to generate PSARNAi-JNK and PSARNA1-PI3K to target the genes of c-Jun N-terminal kinases 1 and 2 (JNK1 and JNK2) and PI3K. Target sites for RNA interference (RNAi) were selected from the human JNK1/JNK2 (5′-GATCAGTGGAATAAAGTTATT-3′; SEQ ID NO: 10), human PI3K (p110β subunit, 5′-AAGCAAGTTCACAATTACCCA-3′; SEQ ID NO:11), and green fluorescence protein (GFP, 5′-TGAAGCAGCACGACTTCTTCA-3′; SEQ ID NO:12).

### RNAi Lentivirus System

The lentiviral construct was modified from pLL3.7 (Rubinson et al. (2003) Nat. Genet. 33: 401-406). In brief, a CMV promoter driving the expression of GFP was deleted. The mouse U6 promoter was replaced with the PSA promoter and enhancer, as shown in the schematic map in FIG. 1A. AATAAA was used as a polyadenylation signal. Lentiviral production was performed as described previously (Lois et al. (2002) Science, 295: 868-872; Tiscoria et al. (2003) Proc. Natl. Acad. Sci. USA, 100: 1844-1848).

### Cell Culture and Transfection

LNCaP cells were grown in RPMI 1640 medium supplemented with 10% fetal bovine serum (Invitrogen, Carlsbad, Calif., USA). HeLa and 293T cells were grown in Dulbecco's modified Eagle's medium supplemented with 10% fetal bovine serum. Transfections were performed using Lipofectamine 2000 according to the manufacturer's protocol (Invitrogen).

### Dot Hybridization

Total RNA was isolated from cells using TRIzol solution (Invitrogen) according to the manufacturer's protocol. Fifty μg total RNA were dotted on a nylon membrane (Bio-Rad, Hercules, Calif.). Radiolabeled 20-mer oligonucleotides of the sense-strand target sequence of JNK were used as a probe. Hybridization was performed as previously described. Song et al. (2004) J. Biol. Chem. 279:24414-24419.

### Western Blot Analyses

Whole-cell lysates were electrophoresed and immunoblotted according to the protocol provided by Santa Cruz Biotechnology, Inc. Anti-JNK1, anti-JNK2, anti-PI3K, and the anti-phospho c-Jun polyclonal antibody were purchased from Santa Cruz Biotechnology, Inc., Santa Cruz, Calif., USA. The anti-c-Jun polyclonal antibody was purchased from Calbiochem., San Diego, Calif., USA, the anti-GFP polyclonal antibody was from BD Bioscience Clontech, Palo Alto, Calif., USA, the anti-FKBP12 polyclonal antibody was from Affinity Bioreagents, Inc., Golden, Colo., USA, and the anti-PARP polyclonal antibody was obtained from Oncogene Res., Boston, Mass., USA.

### TUNEL Staining

Programmed cell death was detected with the In Situ Cell Death Detection Kit, TM red (Roche Applied Science, Indianapolis, Ind., USA). Terminal deoxynucleotidyl transferase-mediated nick end labeling (TUNEL) staining was performed according to the manufacturer's protocol.

### Results and Discussion

To determine whether the PSA promoter is suitable for expressing siRNA, a vector, pPSARNAi-GFP, was developed, utilizing the human PSA promoter and its enhancer to express siRNAs to target the GFP gene, a commonly used indicator (Riegman et al. (1991) Mol. Endocrinol., 5: 1921-1930; and Pang et al. (1997) Cancer Res., 57: 495-499). A 21-mer sequence from the GFP gene was inserted between the PSA promoter and the polyadenylation signal in a pBluescript II KS+vector to generate an siRNA to target GFP (FIG. 1A). In the presence of androgen treatment, the GFP expression plasmid was co-transfected with either pPSARNAi-GFP or empty vector pBluescript II KS+ into the prostate-derived, androgen-responsive LNCaP cell line. Androgen enhances PSA promoter activity due to the androgen-responsive elements (AREs) located in both the PSA promoter and enhancer (Riegman et al. (1991) supra; and Pang et al. (1997) supra). Cervix-derived HeLa cells and kidney-derived 293T cells were used as control cell lines. Forty-eight hours post-transfection, cells were subjected to fluorescence microscopic analysis. The expression of GFP was reduced only in the pPSARNAi-GFP-transfected LNCaP cells, but not in the HeLa or 293T cells, which suggests that PSA expressed an siRNA to silence the target gene in a tissue-specific fashion. Similarly, Western blot analysis further confirmed that inhibition of GFP expression only occurred in pPSARNAi-GFP-transfected LNCaP cells, and not in HeLa or 293T cells (FIG. 1B). Similarly, Western blot analysis further confirmed that inhibition of GFP expression occurred only in pPSARNAi-GFP-transfected LNCaP cells, not in HeLa cells or 293T cells (FIG. 1C).

To determine whether GFP silencing by RNAi in LNCaP cells is androgen-dependent, LNCaP and HeLa cells were each transfected with the aforementioned plasmids in the absence or presence of androgen. Western blot analysis demonstrated that inhibition of GFP expression was observed only in the pPSARNAi-GFP-transfected LNCaP cells treated with androgen (FIG. 1D, lane 4). In contrast, expression of GFP in pPSARNAi-GFP-transfected HeLa cells was unaffected by androgen treatment (FIG. 1D, compare lanes 7 and 8 with lanes 5 and 6). These results suggest that siRNA expression from the PSA promoter is androgen-dependent and tissue-specific.

FIGS. 1A-D. Tissue-specific gene silencing by expression of siRNAs from the human PSA promoter. A, Schamatic map of pPSARNAi-GFP. PSA promoter and enhancer, polyadenylation signal, androgen-responsive element (ARE), transcription start sites (+1 and *), and the target sequence are indicated. B, Western blot analysis of GFP expression. Cell lysates prepared from LNCap, HeLa, and 293T cells that were transfected with empty vector or pPSARNAi-GFP, were subjected to Western blot analysis of GFP expression. Expression of FKBP12 was used as an internal control. C, Western blot analysis of GFP expression. Cell lysates prepared from LNCaP, HeLa, and 293T cells transfected with empty vector or pPSARNAi-GFP were subjected to Western blot analysis of GFP expression. Expression of FKBP12 was used as an internal control. D, Androgen-dependent expression of an siRNA from the PSA promoter. Either pBluescript II KS+vector or pPSARNAi-GFP was cotransfected with the GFP expression plasmid into LNCaP or HeLa cells. The cells were either treated or untreated with 10 nM of androgen. GFP expression was detected by Western blot analysis. Expression of FKBP12 was used as an internal control.

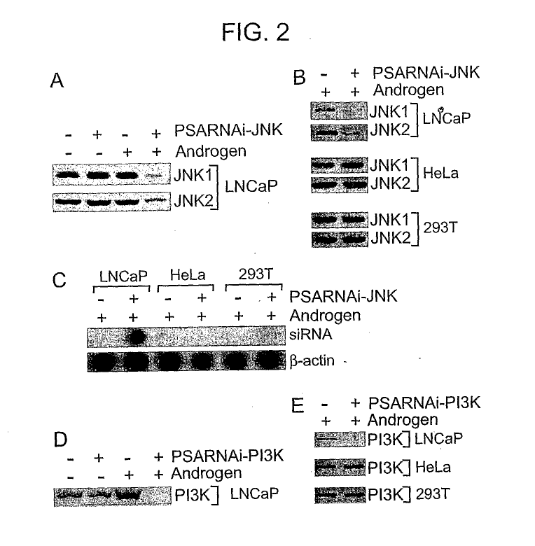

siRNA-mediated endogenous gene silencing from the PSA promoter and enhancer was investigated. To determine whether the knockdown of endogenous genes has a significant impact on the biological functions, signaling regulators such as JNK1 and JNK2, which are involved in controlling cell apoptosis in response to extracellular signaling (Kuan et al. (1999) Neuron, 22: 667-676), were selected. PSARNAi-JNK was constructed in a lentiviral-based vector to silence the human JNK1 and JNK2 genes by virtue of a shared stretch of identical sequence. Forty-eight hours after infecting LNCaP cells with the lentiviral PSARNAi-JNK in the presence or absence of androgen, cell extracts were prepared for Western blot analysis. Significant inhibition of JNK1 and JNK2 was observed only in the androgen-treated lentiviral PSARNAi-JNK-infected LNCaP cells (FIGS. 2A and 2B), suggesting that siRNA expression from the PSA promoter effectively targets endogenous genes and is also androgen-dependent. To investigate whether siRNA-mediated inhibition of JNKs from the PSA promoter is also tissue-specific, LNCaP, HeLa, and 293T cells were infected with lentiviral PSARNAi-JNK in the presence of androgen. Data obtained from Western blot analysis demonstrated that endogenous JNK genes were down-regulated only in LNCaP cells (FIG. 2B), which suggests that siRNA expression from this tissue-specific promoter also leads to endogenous gene silencing in a tissue-specific manner. Similarly, silencing of the endogenous gene, PI3K (Beresford et al. (2001) Cytokine Res. 21:313-322), by lentiviral PSARNA1-PI3K is also androgen-dependent and tissue-specific (FIGS. 2D & 2E).

To determine whether inhibition of JNK resulted from the expression of siRNAs, siRNA expression was examined using dot hybridization. Hybridization signal was detected only in LNCaP cells that were infected with lentiviral PSARNAi-JNK in the presence of androgen (FIG. 2C), whereas no hybridization signals were present in HeLa and 293T cells infected with lentiviral empty vector or lentiviral PSARNAi-JNK (FIG. 2C). These results demonstrate that the inhibition of JNK is dependent on expression of siRNAs. β-Actin was used as a control probe.

FIGS. 2A-E. Androgen-dependent and tissue-specific gene silencing of endogenous genes in LNCaP cells. A, Androgen-dependent knockdown of endogenous JNK from the PSA promoter in LNCaP cells. LNCaP cells were infected with lentiviral PSARNAi-JNK or a lentiviral empty vector with or without androgen treatment. Expression of JNK1 and JNK2 was detected using Western bolt analyses 48 hr post-infection. B, Tissue-specific gene silencing of both human JNK1 and JNK. LNCaP, HeLa, and 293T cells were infected with lentiviral PSARNAi-JNK or lentiviral empty vector and treated with androgen. Forty-eight hours post-infection, cell lysates were subjected to Western blot analysis for the expression of JNK1 and JNK2. C, Expression of siRNAs. Tissue-specific expression of siRNAs was examined using dot hybridization. β-Actin was used as a control. D, Androgen-dependent effect of RNAi in targeting PI3K. LNCaP cells were infected with lentiviral PSARNA1-PI3K or a lentiviral empty vector with or without androgen treatment. Expression of PI3K was detected 48 hr post-infection. E, Tissue-specific gene silencing of human PI3K. LNCaP, HeLa, and 293T cells were infected with either lentiviral PSARNA1-PI3K or control lentivirus in the presence of androgen. Expression of PI3K was analyzed by Western blotting 48 hr post-infection.

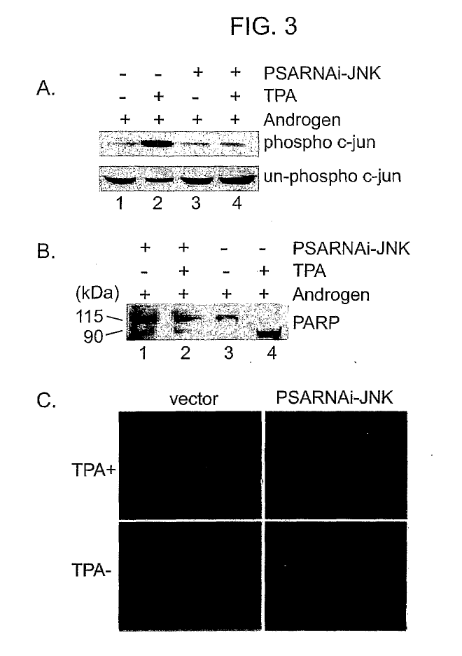

To determine whether JNK-knockdown affects the phosphorylation status of the downstream target c-Jun, 12-O-tetradecanoylphorbol-13-acetate (TPA)-induced phosphorylated c-Jun was used to assess JNK activity. Phosphorylated c-Jun was enhanced in cells infected with a control viral vector (FIG. 3A, lane 2 top panel) but not in cells infected with lentiviral PSARNAi-JNK, even in the presence of TPA treatment (FIG. 3A, lane 4, top panel). Western blot analysis of unphosphorylated c-Jun was used as a control (FIG. 3A, bottom panel). These results clearly demonstrate that the cells with knockdown JNKs lose their responsiveness to TPA-induced JNK activity for phosphorylation of c-Jun, and suggest that siRNA-mediated gene silencing by a tissue-specific promoter has a great impact on the regulation of signaling pathways.

JNK is required for TPA-induced apoptosis in the androgen-responsive prostate cancer cell line, LNCaP (Engedal et al. (2002) Oncogene, 21: 1017-1027). The effect of the JNK gene silencing in TPA-induced apoptosis of LNCaP cells was examined, using the cleaved 90-kD PARP fragment as an apoptosis indicator. As shown in FIG. 3B, lane 4, the detection of the 90-kD PARP indicated that TPA induced apoptosis in empty lentiviral vector-infected LNCaP cells. In contrast, TPA did not enhance the 90-kD PARP fragment in LNCaP cells infected with lentiviral PSARNAi-JNK, suggesting that knockdown JNK prevents cells from undergoing apoptosis in response to TPA treatment (FIG. 3B, compare lane 2 with lane 1). To detect apoptosis at the single cell level, LNCaP cells infected with either lentiviral PSARNAi-JNK or control lentivirus in the presence or absence of TPA treatment were subjected to terminal deoxynucleotidyl transferase (TdT)-mediated dUTP nick and labeling (TUNEL) staining. DNA strand breaks in the cells were then detected by fluorescence microscopy. As shown in FIG. 3C, TPA-treated control cells enhanced TUNEL staining signals. In contrast, knockdown of JNK in LNCaP cells showed a minor stained signal compared to TPA-treated control cells, suggesting that knockdown JNK protects cells from TPA-induced apoptosis in LNCaP cells.

FIGS. 3A-3C. Biological effects of gene silencing of JNKs in LNCaP cells. A, JNKs are required for the phosphorylation of c-Jun when stimulated by TPA. LNCaP cells were infected with lentiviral PSARNAi-JNK or a control lentivirus and treated with androgen. Forty-eight hours post-infection, cells were treated with TPA (100 ng/ml) for one hour prior to harvesting the cells. Expression of phosphorylated and unphosphorylated c-Jun was detected by Western blot analysis. B, Effects of JNK in TPA-induced apoptosis of LNCaP cells. LNCaP cells were infected with lentiviral PSARNAi-JNK or control lentivirus in the presence of androgen treatment. 48 hours after infection, cells were treated with or without TPA (100 ng/ml) for 24 hours, followed by Western blot analysis of PARP expression. C, TUNEL staining. LNCaP cells were cultured in an eight-well chamber slide. 48 hours after infection, cells were treated with or without TPA for an additional 24 hr, followed by TUNEL staining. Images were captured with a digital camera using an Olympus fluorescence microscope.

The data show that siRNA can be expressed from a tissue-specific promoter. This finding indicates that that many superior RNA Pol II-mediated mammalian expression vectors can be used to drive the corresponding small hairpin RNA (shRNA) to silence the targeted gene expression in a tissue-specific manner. Furthermore, an inducible Pol II-mediated expression vector, as described herein, is useful for controlling the expression of shRNA for functional analysis of cell viability-essential genes.

In summary, these data demonstrated that an siRNA expressed from either vector- or lentiviral-based systems using the PSA promoter not only specifically reduced expression of ectopic and endogenous genes in cells, but also acted in a tissue-specific and hormone-dependent manner. The results also demonstrated that inhibition of an apoptosis-related regulatory gene by a tissue-specific PSA promoter altered apoptotic activity. This approach is a cost- and time-effective alternative method to study the function(s) of a targeted gene in cell-based systems and transgenic animals. Further study of the effectiveness of siRNA-mediated gene silencing by the PSA promoter in an animal system will lay the groundwork for creating a potential gene therapy approach for the treatment of prostate cancer.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An isolated nucleic acid comprising, in order from 5′ to 3′ and in operable linkage, a target cell-specific RNA polymerase II promoter, and a nucleotide sequence encoding a short interfering RNA.

2. The nucleic acid of claim 1, further comprising an inducible promoter 5′ of the target cell-specific RNA polymerase II promoter.

3. The nucleic acid of claim 1, wherein the target cell-specific promoter directs transcription in cancer cells.

4. The nucleic acid of claim 3, wherein the cancer cells are prostate cancer cells.

5. The nucleic acid of claim 3, wherein the cancer cells are breast cancer cells.

6. The nucleic acid of claim 3, wherein the siRNA reduces expression of a gene that encodes a product that controls cell proliferation.

7. The nucleic acid of claim 1, wherein the target cell-specific promoter directs transcription in CD4⁺ T cells.

8. The nucleic acid of claim 1, wherein the target cell-specific promoter directs transcription in human immunodeficiency virus-1 (HIV-1)-infected cells.

9. The nucleic acid of claim 7 or claim 8, wherein the siRNA reduces expression of HIV-1.

10. A recombinant expression vector comprising the nucleic acid of claim 1.

11. A composition comprising the recombinant vector of claim 10.

12. A genetically modified host cell comprising the recombinant expression vector of claim 10.

13. A method of reducing expression of a target gene in a target cell, the method comprising introducing the recombinant expression vector of claim 10 into the target cell, wherein the encoded siRNA is specific for the target gene and reduces expression of the target gene.

14. The method of claim 13, wherein the target gene is an endogenous gene.

15. The method of claim 13, wherein the target gene is an exogenous gene.

16. The method of claim 14, wherein the target gene encodes a product that controls cell proliferation.

17. The method of claim 15, wherein the target gene is a gene of an intracellular pathogen.

18. The method of claim 17, wherein the target gene is a viral gene.

19. The method of claim 17, wherein the target cell is a eukaryotic cell.

20. The method of claim 17, wherein the target cell is in vitro.

21. The method of claim 17, wherein the target cell is a prostate cell.
